(19) United States
(12) Patent Application Publication
(10) Pub. No.: US 2009/0306163 A1
(43) Pub. Date: Dec. 10, 2009

(54) TOPICAL COMPOSITIONS COMPRISING IMIDAZOLIDINEDIONE ANALOGS AND THEIR USE TO TREAT OR PREVENT THE APPEARANCE OF SKIN WRINKLING

(76) Inventor: Pamela Renee Lipkin, New York, NY (US)

Correspondence Address: GREENBERG TRAURIG, LLP, 200 PARK AVE., P.O. BOX 677, FLORHAM PARK, NJ, 07932, US

(21) Appl. No.: 12/481,718

(22) Filed: Jun. 10, 2009

Related U.S. Application Data
(60) Provisional application No. 61/060,200, filed on Jun. 10, 2008.

(51) Int. Cl. A61K 31/415

(52) U.S. Cl. 514/390

(57) ABSTRACT

The described invention relates to the formulation and delivery of topical compositions comprising a cosmetically effective amount of at least one imidazolidinedione analog reduce the appearance of wrinkles.

### CROSS REFERENCES

This application claims the benefit of priority to U.S. Application Ser. No. 61/060,200, filed Jun. 10, 2008, herein incorporated in its entirety.

### FIELD OF THE INVENTION

The described invention relates to the formulation and delivery of topical compositions comprising a cosmetically effective amount of at least one imidazolidinedione analog and their use to reduce the appearance of wrinkles.

### BACKGROUND OF THE INVENTION

Facial muscles (also known as musculi facials, or mimetic muscles), are a group of striated muscles innervated by cranial nerve VII, also known as the facial nerve. They are subcutaneous (meaning just under the skin) muscles that control facial expression. They generally originate on bone, and insert on the skin of the face. A “facial expression”, which is a form of nonverbal communication, results from one or more motions or positions of the muscles of the face. The muscles that allow this complex communication are located in superficial positions along the face, including muscles around the eyes, mouth, nose and forehead, the scalp and the neck (Table I.) The largest group of facial muscles is associated with the mouth. Smaller groups of muscles control movements of the eyebrows and eyelids, the scalp, the nose, and the external ear. During a spontaneous smile, for example, the corners of the mouth lift up through movement of the zygomaticus major muscle, and the eyes crinkle, causing “crows feet” through contraction of the orbicularis oculi muscle.

TABLE 1
Muscles of Facial Expression

| Muscle | Origin | Insertion | Action |
|---|---|---|---|
| Frontalis | Galea | Skin of | Raises eyebrows, |
| | aponeurotica | eyebrows and | wrinkles forehead skin |
| | | nose | |
| Orbicularis | Frontal and | Skin of eyelid | Blinking, squinting, |
| oculi | maxillary bone | | forceful closing of |
| | | | eyelids |
| Orbicularis | Fibers of other | Muscles and | Closes and protrudes lips |
| oris | mouth muscles | skin at angle of | |
| | | the mouth | |
| Platysma | Pectoralis and | Lower border | Depresses mandible, |
| | deltoid fascia | of the | draws angle of mouth |
| | | mandible, | downward, tightens |
| | | mouth skin and | skin of the neck |
| | | muscle | |

A “wrinkle” is a ridge or crease of the skin surface caused by the effects of facial muscles. Wrinkling in skin, including, but not limited to, crows feet around the eye, undereye wrinkles, neck wrinkles, “smile lines”, “parentheses lines”, and wrinkles around the lips, is caused by a number of factors, including habitual facial expressions, aging, sun damage, smoking, and poor hydration. Wrinkles can be present as either fine surface lines or deep furrows.

Some subjects will do just about anything to reduce or eliminate the appearance of wrinkles. Consequently, a number of products and procedures have been developed to rejuvenate the appearance of skin. Many of these products and procedures have undesirable side effects.

For example, antioxidants, including preparations that contain vitamins A, C, and E and beta carotene, and ordinary moisturizers affect the appearance of fine lines only temporarily.

Tretinoin (Retin-A™, Avita™, Renova™), a derivative of Vitamin A, generally is believed to be the only topical medication that has been proven to improve wrinkles. It works by increasing skin cell turnover. Known side-effects of tretinoin are darkening of the skin or lack of color temporarily, burning, dry skin, itching, peeling, redness and stinging.

Alpha-hydroxy acids are a class of chemical compounds well-known in the cosmetic industry. They are found in products claiming to reduce wrinkles or the signs of aging, and are used for superficial chemical peels. Known common side effects include mild skin irritation, redness and flaking. Deep chemical peels can have more serious side effects including blistering, burning and skin discoloration, and can increase photosensitivity.

Scarring and pigmentary changes are common complications to microdermabrasion (a procedure that involves gentle “sanding” of the skin); laser resurfacing (whereby lasers are used to peel the skin to the middle of the dermis and to thereby help stimulate the body's natural collagen production), dermabrasion (a surgical procedure often performed under general anesthesia) and chemical peels. Hyaluronic acid fillers, such as Juvederm™ and Restylane™ are injected into the skin to increase volume and flatten wrinkles and folds. Complications may include inflammatory reactions (such as redness, edema, erythema), which may be associated with stinging, pain or pressure at the injection site. Swelling or nodules also may develop at the injection site. Rare cases of necrosis, abcess, granuloma, and hypersensitivity have been described.

BOTOX™, a trade name given to a product containing botulinum toxin, a neurotoxin produced by Clostridium botulinum, is injected through the skin and into a facial muscle. This temporarily paralyzes muscles that produce the “frown lines” on the forehead, fine lines around the eyes, and other wrinkles and may result in a lessening of the appearance of lines and wrinkles. Its effects are temporary (about 2-4 months); therefore repeated treatment is needed. Skin rash, pruritus and allergic reactions, including anaphylaxis, to the toxin have been reported. Localized pain, tenderness and/or burning may be associated with the injection. Weakness of adjacent muscles may occur due to spread of the toxin.

Some subjects turn to surgical facelifts, brow lifts and similar plastic surgery procedures to rejuvenate the appearance of skin,

Therefore, a cosmetic composition that can reduce skin wrinkling without these side effects would be highly desirable.

U.S. Pat. No. 4,049, 650 discloses that 1-[[[5-(substituted phenyl)-2-oxazolyl]methylene]-amino]-2,4-imidazolidinediones having the general formula:

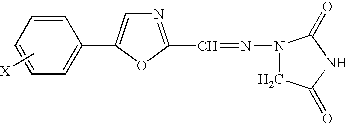

wherein X represents hydrogen, 4-halo, 3,4-dichloro, 4-nitro, 4-methoxy, 4-methyl or 3-trifluoromethyl, have a skeletal muscle relaxant effect when administered to warm-blooded animals.

For example, dantrolene, the sodium salt of (1-[[[5-(4-nitrophenyl)-2-furanyl]methylene]amino][2,4-imidazolidinedione), is an imidazolidinedione wherein X is nitrite. It is available commercially as a muscle relaxant in two forns: as an injectable, intravenous solution, and as capsules for oral administration. Dantrolene is useful to control the manifestations of clinical spasticity resulting from upper neuron disorders, in the prevention and treatment of malignant hyperthermia in humans, and as a cardiac antiarrthymic agent in hypothermic and normothermic warm blooded animals. Upon oral ingestion of dantrolene sodium chemicals, the dantrolene largely is absorbed into the systemic system.

Azumolene, the monosodium salt of [[[5-(4-bromophenyl)-2-oxazolyl]methylene]amino]-2,4-imidazolidinedione, is an imidazolidinedione wherein X is bromo. Azumolene, also a muscle relaxant, has been found to be useful to treat malignant hyperthermia. For treatment of malignant hyperthermic reaction, azmulene is administered parenterally in order to achieve rapid reversal of the malignant hyperthermic reaction. Other routes of administration that result in sufficient blood levels of azumolene also may be used for this purpose.

None of these references teach or suggest topical use of imidazolidinedione derivatives to relax wrinkles.

The topical compositions comprising imidazolidinedione derivatives that are described herein, when applied, for example, around eyelid skin, relax local skin wrinkling without the undesirable side effect of existing treatments.

### SUMMARY OF THE INVENTION

In one aspect, the described invention provides a topical composition to treat or prevent skin wrinkling comprising a cosmetically effective amount of a compound of formula I or a salt of Formula I wherein R¹, R², R³, R⁴, and R⁵ are each independently H, alkyl, cycloalkyl, alkylcycloalkyl, alkylaryl, alkylheteroalkyl; A, B and C are each independently C, N, S, O, SO or SO₂; X═CH═CH; C, NR⁵, O, S or a bond to the phenyl ring; Yₙ is halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, N⁴, NR⁵, CONR¹, CONR², CONR³, CONR⁴, CONR⁵; n=0, 1, 2, 3, 4, or 5; Zₘ=halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵; and m=0, 1, 2, or 3 and a carrier. According to one embodiment of the composition, the compound of formula I is dantrolene. According to another embodiment, the compound of formula I is azumolene. According to another embodiment, the composition is applied to the skin around at least one of an eye, a mouth, a nose, a forehead, a scalp, a decolletage, and a neck. According to another embodiment, the composition further includes an additional active ingredient. According to some such embodiments, the additional active ingredient is selected from the group consisting of a protective agent, an emollient, an irritant, a keratolytic, a sun screening agent, a sun tanning agent, a peptide, a niacinamide, farnesol, phytantriol, salicylic acid, hydroxy acid, an anti-cellulite agent, bisabolol, an antibiotic agent, an antifungal agent, an antiviral agent, an antiprotozoal agent, an anti-acne agent, a steroidal anti-inflammatory agent, a non-steroidal anti-inflammatory agent, an antipruritic agent, an anti-oxidant agent, an anti-histamine agent, a vitamin, an anti-wrinkle agent, an anti-skin atrophy agent, a sclerosing agent, a caustic agent and a hypo-pigmenting agent, or combinations thereof According to another embodiment, the composition is formulated as a cream. According to another embodiment, the composition is formulated as a lotion. According to another embodiment, the composition is formulated as an ointment. According to another embodiment, the composition is formulated as a gel.

In another aspect, the described invention provides a method of treating or preventing wrinkling of skin, the method comprising the step of topically applying onto an epithelial surface of a subject, including a human, in need thereof, a cosmetically effective amount of a composition comprising a compound of Formula I or a salt of formula I, wherein R¹, R², R³, R⁴, and R⁵ are each independently H, alkyl, cycloalkyl, alkylcycloalkyl, alkylaryl, alkylheteroalkyl; A, B and C are each independently C, N, S, O, SO or SO₂; X═CH═CH; C, NR⁵, O, S or a bond to the phenyl ring; Yₙ is halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵; n=0, 1, 2, 3, 4, or 5; Zₘ=halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵; m=0, 1, 2, or 3; and a carrier thereby treating or preventing wrinkles. According to one embodiment of the method, the compound of formula I is dantrolene. According to another embodiment, the compound of formula I is azunolene. According to another embodiment, the epithelial surface onto which the composition is applied topically is a face. According to another embodiment, the epithelial surface onto which the composition is applied topically comprises skin around an eye. According to another embodiment, the epithelial surface onto which the composition is applied topically comprises an eyelid. According to another embodiment, the epithelial surface onto which the composition is applied topically comprises skin around a mouth. According to another embodiment, the epithelial surface onto which the composition is applied topically comprises a forehead. According to another embodiment, the epithelial surface onto which the composition is applied topically comprises a decolletage. According to another embodiment, the epithelial surface onto which the composition is applied topically comprises a neck. According to another embodiment, the composition fuirther includes an additional active ingredient. According to some such embodiments, the additional active ingredient is selected from the group consisting of a protective agent, an emollient, an irritant, a keratolytic, a sun screening agent, a sun tanning agent, a peptide, a niacinamide, farnesol, phytantriol, salicylic acid, hydroxy acid, an anti-cellulite agent, bisabolol, an antibiotic agent, an antifungal agent, an antiviral agent, an antiprotozoal agent, an anti-acne agent, a steroidal anti-inflammatory agent, a non-steroidal anti-inflammatory agent, an antipruritic agent, an anti-oxidant agent, an anti-histamine agent, a vitamin, an anti-wrinkle agent, an anti-skin atrophy agent, a sclerosing agent, a caustic agent and a hypo-pigmenting agent, or combinations thereof According to another embodiment, the composition is formulated as a cream. According to another embodiment, the composition is formulated as a lotion. According to another embodiment, the composition is formulated as an ointment. According to another embodiment, the composition is formulated as a gel.

### DETAILED DESCRIPTION

The compositions of the described invention comprise (a) at least one compound of Formula (I) or a salt of Formula (I):

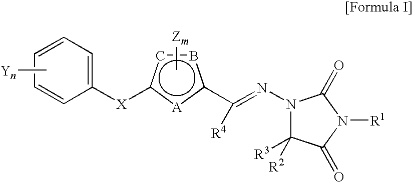

wherein
R¹, R², R³, R⁴, and R⁵ are each independently H, alkyl, cycloalkyl, alkylcycloalkyl, alkylaryl, alkylheteroalkyl;
A, B and C are each independently C, N, S, O, SO or SO₂;
X═CH═CH; C, NR⁵, O, S or a bond to the phenyl ring;
Yₙ is halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵; n=0, 1, 2, 3, 4, or 5;
Zₘ=halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵; and m=0, 1, 2, or 3.

In some embodiments, compositions of the described invention comprise at least one imidazolidinone compound as shown in Table 2.

TABLE 2
Imidazolidinone Compounds

| | R¹ | R² | R³ | R⁴ | R⁵ | A |
|---|---|---|---|---|---|---|
| Dantrolene | H | H | H | H | — | O |
| Azumolene | H | H | H | H | — | O |
| U.S. Pat. No. | H, hydroxyethyl, butyl, | H | H | H | — | O |
| 3,415,821 | 4-pyridylethyl | | | | | |
| U.S. Pat. No. | H | H | H | H | — | O |
| 4,049,650 | | | | | | |
| U.S. Pat. No. | H | H | H | H | — | O |
| 4,822,629 | | | | | | |
| U.S. Pat. No. | H | H | H | H | — | O |
| 4,861,790 | | | | | | |
| Hosoya (Bioorganic & Medicinal Chem. 11: 663-73 (2003)) | 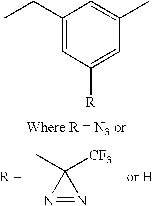 | H | H | H | — | O |
| Ikemoto (Br. J. Pharmcol. 134: 729-36 (2001)) | 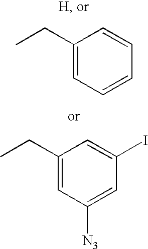 | 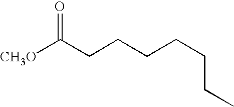 | H | H | — | O |
| Zhao, J Biol. Chem. 281 | H | H | H | H | — | O |
| (44): 33477-86 (2006)) | | | | | | |
| Zhang, J. Pharmacol. | H | H | H | H | — | O |
| & Exptl Therapeutics | | | | | | |
| 314 (1): 94-102 (2005)) | | | | | | |
| Leslie, Br. J. Pharmacol. | H | H | H | H | — | O |
| 97: 1151-56 (1989)) | | | | | | |
| Hosoya (Bioorganic & Medicinal Chemistry Letters 1: 3263-65 (2002 | 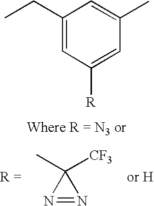 | H | H | H | — | O |
| Salinska (Neurosci | H | H | H | H | — | O |
| Leters (2008) doi: 10, | | | | | | |
| 1016 (j.neulet.2007. | | | | | | |
| 12.013))) | | | | | | |
| Snyder (J. Medicinal Chem. 10: 807-10 (1967) | H, Na, CH₂CH₂OH; (CH₂)₃CH₃; CH₂CH₂-4-C₅H₄N | H | H | H | — | O |

| | B | C | X | Y | n | Z | m |
|---|---|---|---|---|---|---|---|
| Dantrolene | C | C | Bond | NO₂ | 1 | — | 0 |
| Azumolene | N | C | Bond | Br | 1 | — | 0 |
| U.S. Pat. No. | C | C | Bond | Nitro, cyano, amino, | 1 or 2 | — | 0 |
| 3,415,821 | | | | chloro, bromo, acetyl, | | | |
| | | | | carboxy, methyl, | | | |
| | | | | trifluoromethyl and H | | | |
| U.S. Pat. No. | N | C | bond | H, 4-Halo; 3,4-Chloro; | 1 or 2 | — | 0 |
| 4,049,650 | | | | 4-nitro; 4-methoxy; | | | |
| | | | | 4-methyl; 3-CF₃ | | | |
| U.S. Pat. No. | N | C | Bond | Br | 1 | — | 0 |
| 4,822,629 | | | | | | | |
| U.S. Pat. No. | N | C | Bond | Br | 1 | — | 0 |
| 4,861,790 | | | | | | | |
| Hosoya (Biorganic & Medicinal Chem. 11: 663-73 (2003)) | 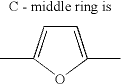 | C | Bond | NO₂, 4-CH₃O; 3-CH₃O; 2-CH₃O, H; 4-CH₃; 4-NO₂; 3-NO₂; 2-NO₂; 4-CF₃; 2,3,4,5,6-F₅; 4-F; 4-C₆H₅; 2,6-(NO₂)₂; 2,3-Benzo; 4-Br; 4-OH; 4-CF₃SO₂; 4-CN | 1 | — | 0 |
| Ikemoto (Br. J. | N | C | Bond | NO₂, I, OCH₃ | 1 | — | 0 |
| Pharmcol. 134: 729-36 | | | | | | | |
| (2001)) | | | | | | | |
| Zhao, J Biol. Chem. 281 | N | C | Bond | Br | 1 | — | 0 |
| (44): 33477-86 (2006)) | | | | | | | |
| Zhang, J. Pharmacol. | N | C | Bond | Br | 1 | — | 0 |
| & Exptl Therapeutics | | | | | | | |
| 314 (1): 94-102 (2005)) | | | | | | | |
| Leslie, Br. J. Pharmacol. | N | C | Bond | Br | 1 | — | 0 |
| 97: 1151-56 (1989)) | | | | | | | |
| Hosoya (Bioorganic & Medicinal Chemistry Letters 1: 3263-65 (2002 | 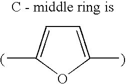 | C | BOND | 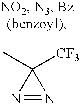 | 1 | — | 0 |
| Salinska (Neurosci | N | C | Bond | NO₂ | 1 | — | 0 |
| Leters (2008) | | | | | | | |
| doi: 10, 1016 | | | | | | | |
| (j.neulet.2007.12.013))) | | | | | | | |
| Snyder (J. Medicinal Chem. 10: 807-10 (1967) | 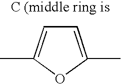 | | | C₆H₅; C₆H₄NO₂-p; C₆H₄NO₂-m; C₆H₄NO₂-o; C₆H₃CH₃-o-NO₂-p; C₆H₃Cl-o-NO₂-p; C₆H₄Cl-p; C₆H₃Cl₂-m,p; C₆H₃Cl-o-CF₃-m; C₆H₄Br-p; C₆H₄F-p; C₆H₃CF₃-m; C₆H₄CN-p; C₆H₄COCH₃-p; C₆H₄CO₂H-p; C₆H₄NH₂-p; 2-C₄H₃O | | | |

The term “aliphatic” as used herein, denotes a straight- or branched-chain arrangement of constituent carbon atoms, including, but not limited to paraffins (alkanes), which are saturated, olefins (alkenes or alkadienes), which are unsaturated, and acetylenes (alkynes), which contain a triple bond. In complex structures, the chains may be branched or cross-linked.

The term “alkyl,” as used herein, denotes a straight (unbranched) or branched univalent aliphatic group of about 1 to about 25 carbon atoms including, but not limited to, methyl, ethyl, propyl, isopropyl, decyl, undecyl, dodecyl, octadecyl, nonadecyl, eicosyl, heneicosyl, decosyl, tricosyl, tetracosyl, and pentacosyl, and the branched (non-straight-chained) isomers thereof, with multiple degrees of substitution being allowed.

The term “alkenyl,” as used herein, denotes a monovalent, straight (unbranched) or branched hydrocarbon chain having one or more double bonds therein where the double bond can be unconjugated or conjugated to another unsaturated group (e.g., a polyunsaturated alkenyl) and can be unsubstituted or substituted, with multiple degrees of substitution being allowed. For example, and without limitation, the alkenyl can be vinyl, allyt, butenyl, pentenyl, hexenyl, butadienyl, pentadienyl, hexadienyl, 2-ethylhexenyl,2-propyl-2-butenyl,4-(2-methyl-3-butene)-pentenyl, decenyl, undecenyl, dodecenyl, heptadecenyl, octadecenyl, nonadecenyl, eicosenyl, heneicosenyl, docosenyl, tricosenyl, tetracisenyl, pentacosenyl, phytyl, the branched chain isomers thereof, and polyunsaturated alkenes including octadec-9,12,-dienyl, octadec-9,12,15-trienyl, and eicos-5,8,11,14-tetraenyl.

As used herein, the term “aryl” refers to a benzene ring or to an optionally substituted benzene ring system fused to one or more optionally substituted benzene rings, with multiple degrees of substitution being allowed. Examples of aryl include, but are not limited to, phenyl, 2-napthyl, 1-naphthyl, 1-anthracenyl, and the like.

It should be understood that wherever the terms “alkyl” or “aryl” or either of their prefix roots appear in a name of a substituent, they are to be interpreted as including those limitations given above for alkyl and aryl.

As used herein, the terms “carbarnyl” “carbamates” or “urethanes” refer to a group of organic compounds sharing a common functional group having the general structure —NH(CO)O—.

As used herein, the terms “cycloalkyl” or “aliphatic cyclic” are used interchangeably to refer to an alicyclic hydrocarbon group optionally possessing one or more degrees of unsaturation, having from three to twelve carbon atoms, optionally substituted with substituents, with multiple degrees of substitution being allowed. “Cycloalkyl” includes, but is not limited to, cyclopropyl, cyclobutyl, cyclopentyl, cyclohexyl, cycloheptyl, cyclooctyl, and the like.

As used herein, the terms “heterocycle” and “heterocyclic” are used interchangeably to refer to a three to twelve-membered heterocyclic ring optionally possessing one or more degrees of unsaturation, containing one or more heteroatomic substitutions selected from —S—, —SO—, —SO₂—, —O—, or —N—, optionally substituted with substitutents, including, but not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring optionally may be fused to one or more of another “heterocyclic” ring(s). Examples of “heterocyclic” include, but are not limited to, pyrrole, furan, thiophene, imidazole, oxazole, thiazole, pyrazole, 3-pyrroline, pyrrolidine, pyridine, pyrimidine, purine, quinoline, isoquinoline, carbazole and the like.

The term C-linked heterocycle means a heterocycle that is bonded through a carbon atom, e.g. —(CH₂)ₙ-heterocycle where n is 1, 2 or 3 or —C<heterocycle where C< represents a carbon atom in a heterocycle ring. Similarly, R moieties that are N-linked heterocycles mean a heterocycle that is bonded through a heterocycle ring nitrogen atom, e.g. —N<heterocycle where N< represents a nitrogen atom in a heterocycle ring. A variable group such as an R moiety that is bonded to a formula 1 compound can be a C-linked heterocycle or an N-linked heterocycle, These heterocycles include those listed below or described elsewhere herein.

Examples of heterocycles include by way of example and not limitation pyridyl, thiazolyl, tetrahydrothiophenyl, sulfr oxidized tetrahydrothiophenyl, pyrimidinyl, fuiranyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyt, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathiinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, .beta.-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuelidinyl, morpholinyl, oxazolidinyl, benzotriazolyl, benzisoxazolyl, oxindolyt, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at the nitrogen atom or position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or .beta.-carboline. Typically, nitrogen bonded heterocycles include 1-aziridyl, 1-azetedyl, 1-pyrrolyl, 1-imidazolyl, 1-pyrazolyl, and 1-piperidinyl and structures such as and tautomers of any of these.

“Heteroaryl” means an aromatic ring or two or more fused rings that contain one or more aromatic rings where the ring or fused rings comprise 1, 2, 3 or more heteroatoms, usually oxygen (—O—), nitrogen (—NX—) or sulfur (—S—) where X is —H, a protecting group or optionally substituted alkyl. Examples are as described for heterocycle.

As used herein the term “isomer” refers to one of two or more molecules having the same number and kind of atoms and hence the same molecular weight, but differing in respect to the arrangement or configuration of the atoms. Generally, differences in compounds having identical molecular and chemical formulas but differing in the way their atoms are arranged three-dimensionally in space create optically active isomers or enantiomers. These enantiomers are called (D-(for dexorotatory, right-handed or +) or L-(levorotatory, left-handed, or −) for the direction in which they rotate plane-polarized light. A mixture of equal parts of the optical forms of a compound is known as a racemic mixture or racemate. A racemic mixture is optically inactive, i.e., it consists of equal parts of D- and L-enantiomers.

The term “O-linked moiety” means a moiety that is bonded through an oxygen atom. Thus, when an R group is an O-linked moiety, that R is bonded through oxygen and it thus can be an ether, an ester (e.g., —O—C(O)-optionally substituted alkyl), a carbonate or a carbamate (e.g., —O—C(O)—NH₂ or —O—C(O)—NH-optionally substituted alkyl). Similarly, the term “S-linked moiety” means a moiety that is bonded through a sulfur atom. Thus, when an R group is an S-linked moiety, that R is bonded through sulfur and it thus can be a thioether (e.g., —S-optionally substituted alkyl), a thioester (—S—C(O)-optionally substituted alkyl) or a disulfide (e.g., —S—S-optionally substituted alkyl). The term “N-linked moiety” means a moiety that is bonded through a nitrogen atom. Thus, when an R group is an N-linked moiety, the R group is bonded through nitrogen and one or more of these thus can be an N-linked amino acid such as —NH—CH₂—COOH, a carbamate such as —NH—C(O)—O-optionally substituted alkyl, an amine such as —NH-optionally substituted alkyl, an amide such as —NH—C(O)-optionally substituted alkyl or —N₃. The term “C-linked moiety” means a moiety that is bonded through a carbon atom. When one or more R group is bonded through carbon, one or more of these thus can be -optionally substituted alkyl such as —CH₂—CH₂—O—CH₃, C(O)-optionally substituted alkyl hydroxyalkyl, mercaptoalkyl, aminoalkyl or ═CH-optionally substituted alkyl.

In some embodiments, the composition of the described invention comprises at least one imidazolidinedione analog. In some embodiments, the at least one imidazolidinedione analog of the composition of the described invention is dantrolene. In some embodiments, the at least one imidazolidinedione analog of the composition of the described invention is azumolene. In another aspect of the described invention, two or more imidazolidinedione analogs are used in the inventive composition to obtain a specific pharmaceutical or cosmeticeutical effect.

The described invention offers compositions useful for treating a condition of the skin. The phrase “epithelia” or “epithelial” or “epithelial tissues” as used herein is meant to refer to skin. Human skin is responsible, inter alia, for protection of the body, regulation of body temperature, sensory reception, synthesis of vitamins and hormones, water balance and absorption of substances. It is a formidable barrier to the passage of most substances. Skin consists of two main parts. an epithelium, the epidermis, lying outermost, and beneath this a layer of connective tissue, which includes the tough collagen-rich dermis and the underlying fatty subcutaneous layer or hypodermis.

The defining component of the skin is the epidermis, which is a multilayered (“stratified”) epithelium composed largely of keratinocytes that synthesize keratins, which give the epidermis its toughness. The waterproof barrier is formed by epidermal cells. The outer, horny layer of the epidermis is the stratum corneum, which consists of several layers of flat keratinized nonnucleated cells. The cell envelopes of the cells in the stratum corneum tend to be mainly polar lipids, such as ceramides, sterols, and fatty acids, while the cytoplasm of stratum corneum cells remains polar and aqueous. Despite the close packing of the cells, about 15% of the stratum corneum is intercellular and, generally lipid-based. The lipid-rich intercellular space in the stratum corneum comprises lamellar matrices with alternating hydrophilic layers and lipophilic bilayers formed during the process of keratinization.

The dermis comprises a fibrous protein matrix embedded in an amorphous, colloidal, ground substance. It supports and interacts with the epidermis, facilitating its conformation to underlying muscles and bones. Blood vessels, lymphatics, and nerves are found within the dermis.

Cutaneous administration involves “topical administration” or “topically applying. The term “topical” refers to administration of an inventive composition at, or immediately beneath, the point of application. The phrase “topically applying” refers to direct application to the area of the surface to be affected. The composition may be applied by pouring, dropping, or spraying, if a liquid; rubbing on, if an ointment, lotion, cream, gel, or the like; dusting, if a powder; spraying, if a liquid or aerosol composition; or by any other appropriate means.

Substances are applied to the skin to elicit one or more of four general effects: an effect on the skin surface, an effect within the stratum corneum; an effect requiring penetration into the epidermis and dermis; or a systemic effect resulting from delivery of sufficient amounts of a given substance through the epidermis and the dermis to the vasculature to produce therapeutic systemic concentrations. One example of an effect on the skin surface is formation of a film. Film formation may be protective (e.g., sunscreen) and/or occlusive (e.g., to provide a moisturizing effect by diminishing loss of moisture from the skin surface). One example of an effect within the stratum corneum is skin moisturization which may involve the hydration of dry outer cells by surface films or the intercalation of water in the lipid-rich intercellular laminae; the stratum corneum also may serve as a reservoir phase or depot wherein topically applied substances accumulate due to partitioning into, or binding with, skin components.

It generally is recognized that short-term penetration occurs through the hair follicles and the sebaceous apparatus of the skin, while long term penetration occurs across cells. Penetration of a substance into the viable epidermis and dermis may be difficult to achieve, but once it has occurred, the continued diffusion of the substance into the dermis is likely to result in its transfer into the microcirculation of the dermis and then into the general circulation. It is possible, however, to formulate delivery systems that provide substantial localized delivery.

“Percutaneous absorption” is the absorption of substances from outside the skin to positions beneath the skin, including into the blood stream. The epidermis of human skin is highly relevant to absorption rates. Passage through the stratum corneum marks the rate-limiting step for percutaneous absorption. The major steps involved in percutaneous absorption of, for example, a drug substance, include the establishment of a concentration gradient, which provides a driving force for drug movement across the skin, the release of drug from the vehicle into the skin-partition coefficient, and drug diffusion across the layers of the skin-diffusion coefficient. The relationship of these factors to one another is summarized by the following equation:

J=Cveh×Km.D/x [Formula 2]

where J=rate of absorption; Cveh=concentration of drug in vehicle; Km=partition coefficient; and D=diffusion coefficient.

There are many factors that affect the rate of percutaneous absorption of a substance. Primarily they are as follows: (i) Concentration. The more concentrated the substance, the greater the absorption rate. (ii) Size of skin surface area. The wider the contact area of the skin to which the substance is applied, the greater the absorption rate. (iii) Anatomical site of application. Skin varies in thickness in different areas of the body. A thicker and more intact stratum corneum decreases the rate of absorbency of a substance. The stratum corneum of the facial area is much thinner than, for example, the skin of the palms of the hands. The facial skin's construction and the thinness of the stratum corneum provide an area of the body that is optimized for percutaneous absorption to allow delivery of active agents both locally and systemically through the body. (iv) Hydration. Hydration (meaning increasing the water content of the skin) causes the stratum corneum to swell which increases permeability. (v) Skin temperature. Increased skin temperature increases permeability. (vi) Composition. The composition of the compound and of the vehicle also determines the absorbency of a substance.

Factors that determine the choice of vehicle and the transfer rate across the skin are the substance's partition coefficient, molecular weight and water solubility. The protein portion of the stratum corneum is most permeable to water soluble substances and the lipid portion of the stratum corneum is most permeable to lipid soluble substances. It follows that substances having both lipid and aqueous solubility may traverse the stratum corneum more readily. Permeability experiments have shown that the hydrated stratum corneum has an affinity for both lipophilic and hydrophilic compounds. This bifunctional solubility arises from the hydrophilic cells and the lipid rich lamellar structures in the intercellular space. Thus, attempts to predict permeability constants from oil: water or solvent: water partition coefficients have had limited success, and for any specific molecule, the predictability of regional variations in skin permeability continues to elude investigators.

Most substances applied topically are incorporated into bases or vehicles. The vehicle chosen for a topical application will greatly influence absorption, and itself may have a beneficial effect on the skin. Ideally, a vehicle has use in the present invention if it is easy to apply and remove, nonirritating, and cosmetically pleasing.

In one embodiment, the composition of the described invention is a pharmaceutical composition. As used herein, a “pharmaceutical composition” refers to a composition that is employed to prevent, reduce in intensity, cure or otherwise treat a target condition or disease.

In another embodiment, the composition of the invention is a cosmetic composition. As used herein a “cosmetic composition” refers to a composition that is intended to be rubbed, poured, sprinkled, or sprayed on, introduced into, or otherwise applied to a subject or any part thereof for beautifying, promoting attractiveness, or altering the appearance, or an article intended for use as a component of any such article, except that such term does not include soap.

In another embodiment, the composition of the invention is a cosmeceutical composition. As used herein the term “cosmeceutical composition” refers to a composition that is employed as both a cosmetic composition and as a pharmaceutical composition.

In another aspect of the described invention, the composition of the described invention includes a carrier. As used herein “carrier” describes a material that does not cause significant irritation to a subject's skin and does not abrogate the biological activity and properties of the imidazolidinedione analog compound of the composition of the described invention. Carriers must be of sufficiently high purity and of sufficiently low toxicity to render them suitable for administration to the mammal being treated. The carrier can be inert or it can possess pharmaceutical benefits, cosmetic benefits or both.

Some non-limiting representative examples of carriers include moisturizing agents or humectants, pH adjusting agents, fragrances, chelating agents, preservatives, emulsifiers, thickeners, solubilizing agents, penetration enhancers, anti-irritants, colorants and surfactants.

As used herein a “moisturizing agent” is a substance that adds or restores moisture to the skin. Representative examples of moisturizing or humectant agents that are usable in the described invention include, without limitation, guanidine, glycolic acid and glycolate salts (e.g. ammonium salt and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

As is widely recognized in the art, since the pH of the skin is 5.5, compositions for topical skin application (to avoid irritation) should have a pH value of between 5.0 and 7.0, between 5.0 and 6.0, about 5.5 or substantially 5.5. Hence, a pH adjusting composition is typically added to bring the pH of the composition to the desired value. The compositions of the described invention therefore may be formulated to have a pH value that ranges between about 5.0 and about 7.0, or about 5.0 and about 6.0. Suitable pH adjusting agents include, for example, but are not limited to, one or more adipic acids, glycines, citric acids, calcium hydroxides, magnesium aluminometasilicates, buffers or any combinations thereof.

As used herein “fragrance” refers to a substance having a pleasant aroma, Suitable fragrances include, but are not limited to, eucalyptus oil, camphor synthetic, peppermint oil, clove oil, lavender, chamomile and the like.

Chelating agents are optionally added to the compositions of the described invention so as to enhance the preservative or preservative system. Preferred chelating agents are mild agents, such as, for example, ethylenediaminetetraacetic acid (EDTA), EDTA derivatives, or any combination thereof.

Suitable preservatives for use in the compositions of the present composition include, without limitation, one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or any combinations thereof.

“Emulsifiers” as used herein promote the formation and stabilization of an emulsion. Suitable emulsifiers may be natural materials, finely divided solids, or synthetic materials. Natural emulsifying agents may be derived from either animal or vegetable sources. Those from animal sources include gelatin, egg yolk, casein, wool fat, or cholesterol. Those from vegetable sources include acacia, tragacanth, chondrus, or pectin. Vegetable sources specifically from cellulose derivatives include methyl cellulose and carboxymethyl cellulose to increase the viscosity. Finely divided emulsifiers include bentonite, magnesium hydroxide, aluminum hydroxide, or magnesium trisylicate. Synthetic agents include anionic, cationic or nonionic agents. Particularly useful are sodium lauryl sulfate, benzalkonium chloride or polyethylene glycol 400 monostearate, or any combinations thereof

“Thickeners” as used herein refer to agents that make the composition of the described invention dense or viscous in consistency. Suitable thickeners that can be used in the context of the described invention include, for example, non-ionic water-soluble polymers such as hydroxyethylcellulose (commercially available under the Trademark Natrosol™ 250 or 350), cationic water-soluble polymers such as Polyquat 37 (commercially available under the Trademark Synthalen™ CN), fatty alcohols, fatty acids, anionic polymers, and their alkali salts and mixtures thereof.

As used herein “solubilizing agents” are those substances that enable solutes to dissolve. Representative examples of solubilizing agents that are usable in the context of the described invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWYEEN® and Spans, e.g., TWEEN 80® and Span20®. Other solubilizers that are usable for the compositions of the described invention are, for example, polyoxyethylene sorbitan fatty acid ester, polyoxyethylene n-alkyl ethers, n-alkyl amine n-oxides, polyoxamers, organic solvents, such as acetone, phospholipids and cyclodextrins.

A “penetration enhancer” is an agent known to accelerate the delivery of a substance through the skin. Suitable penetration enhancers usable in the described invention include, but are not limited to, dimethylsulfoxide (DMSO), dimethyl formamide (DMF), allantoin, urazole, N,N-dimethylacetamide (DMA), decylmethylsulfoxide (C₁₀ MSO), propylene glycol caprylate, polyethylene glycol monolaurate (PEGML), propylene glycol (PG), propylene glycol monolaurate (PGML), glycerol monolaurate (GML), lecithin, polyoxylglycerides, such as Labrafil® (oleolyl macrogolglycerides) and Labrasol® (caprylocaproyl macroglolgycerides); Transcutol®P (diethylene glycol monoethyl ether), the 1-substituted azacycloheptan-2-ones, particularly 1-n-dodecylcyclazacycloheptan-2-one (available under the trademark Azone™ from Whitby Research Incorporated, Richmond, Va.), alcohols, and the like. The permeation enhancer also may be a vegetable oil. Such oils include, for example, safflower oil, cottonseed oil and corn oil.

Additional thickeners, penetration enhancers and other adjuvants may generally be found in Remington's Pharmaceutical Sciences, 18^{th} or 19^{th} editions, published by the Mack Publishing Company of Easton, Pa. which is incorporated herein by reference.

As used herein, an “anti-irritant” is an agent that prevents or reduces soreness, roughness, or inflammation of a bodily part. Presently known anti-irritants may be divided into water-soluble anti-irritants and water-insoluble anti-irritants. Representative examples of such compositions are described, for example, in U.S. Pat. No. 5,482,710 which is herein incorporated by reference. Suitable anti-irritants that may be used in the context of the described invention include, for example, steroidal and non steroidal anti-inflammatory agents or other materials such as aloe vera, chamomile, alpha-bisabolol, cola nitida extract, green tea extract, tea tree oil, licorice extract, allantoin, caffeine or other xanthines, glyeyrrhizic acid and its derivatives.

Colorants also may be used in the compositions of the described invention. Colorants include pigments or dyes or a combination thereof as the cosmetic benefit requires. Suitable pigments include, but are not limited to, iron oxides, and titanium oxides. Suitable dyes include FD&C approved colorants, D&C approved colorants, and those approved for use in Europe and Japan. See Marmion, D. M., Handbook of US Colorants for Food, Drugs, Cosmetics, and Medical Devices, 3rd ed. 1991 herein incorporated by reference.

The term “surfactants” as used herein refers to surface-active substances, such as a detergent. Suitable surfactants for use with the inventive compositions include, but are not limited to, sarcosinates, glutamates, sodium alkyl sulfates, ammonium alkyl sulfates, sodium alkyleth sulfates, ammonium alkyleth sulfates, ammonium laureth-n-sulfates, sodium laureth-n-sulfates, isothionates, glycerylether sulfonates, sulfosuccinates and combinations thereof where an anionic surfactant is desired, suitable anionic surfactants that may be used include, but are not limited to, sodium lauroyl sarcosinate, monosodium lauroyl glutamate, sodium alkyl sulfates, ammonium alkyl sulfates, sodium alkyleth sulfates, ammonium alkyleth sulfates, and combinations thereof.

In some embodiments, the composition of the described invention contains a pharmaceutically acceptable carrier. As used herein “a pharmaceutically acceptable carrier” is any substantially non-toxic carrier conventionally useable for topical administration of pharmaceuticals in which the imidazolidinedione analog compound will remain stable and bioavailable when applied directly to skin or mucosal surfaces.

Suitable pharmaceutically acceptable carriers include water, petroleum jelly (Vaseline™), petroleum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, alcohols, polyols, and the like. Also included are the carriers described hereinabove.

In another embodiment, the pharmaceutically acceptable carrier of the composition of the described invention includes a sustained release or delayed release carrier. The carrier can be any material capable of sustained or delayed release of the imidazolidinedione analog compound to provide a more efficient administration resulting in less frequent and/or decreased dosage of the imidazolidinedione analog compound, ease of handling, and extended or delayed effects on epithelial-related conditions. Non-limiting examples of such carriers include liposomes, microsponges, microspheres, or microcapsules of natural and synthetic polymers and the like. Liposomes, which may enhance the localized delivery of the compounds of the inventive composition within skin layers, may be formed from a variety of phospholipids, such as cholesterol, stearylamines or phosphatidylcholines.

In some embodiments, the compositions of the described invention include a cosmetically acceptable carrier. As used herein the phrase “cosmetically acceptable carrier” refers to a substantially non-toxic carrier, conventionally useable for the topical administration of cosmetics, with which the imidazolidinedione analog compounds will remain stable and bioavailable. It will be understood that cosmetically acceptable carriers and pharmaceutically acceptable carriers are similar, if not often identical, in nature. Suitable cosmetically acceptable carriers are described in the CTFA International Cosmetic Ingredient Dictionary and Handbook, 8th edition, edited by Wenninger and Canterbery, (The Cosmetic, Toiletry, and Fragrance Association, Inc., Washington, D.C., 2000), which is herein incorporated by reference. Also included are the carriers described hereinabove.

Topical skin care product compositions often contain a variety of polymeric substances, natural and/or synthetic, for the purpose of delivering active ingredients to the surface of the skin and/or providing moisturization of the skin. Hyaluronan or hyaluronic acid (HA) is a naturally occurring polysaccharide that consists of alternating N-acetyl-D-glucosarnine and D-glucuronic acid monosaccharide units linked with alternating [beta]1-3 glucoronidic and [beta]1-4 glucosaminidic bonds. The molecular weight of hyaluronic acid is generally within the range of 50,000 up to more than 8×10⁶ Daltons. Cross-linked HA gels include, but are not limited to, hylan B gel matrix, which is a vinyl sulfone cross-linked derivative of hyaluronic acid. Cross-linked simple and mixed gels based on hyaluronan, products containing such gels, and methods to prepare same are described in U.S. Pat. Nos. 4,582,865, 4,605,691, 4,636,524, the contents of which are incorporated by reference herein. It should be understood that hyaluronan of any origin may be successfully used for the purpose of the described invention.

In another embodiment the compositions of the described invention may further include one or more additional compatible active ingredients which are aimed at providing the composition with another pharmaceutical, cosmeceutical or cosmetic effect, in addition to that provided by the imidazolidinedione analog compound of the inventive composition. “Compatible” as used herein means that the components of such a composition are capable of being combined with each other in a manner such that there is no interaction that would substantially reduce the efficacy of the composition under ordinary use conditions.

In one embodiment, the imidazolidinedione analog compound of the inventive compositions is an active ingredient

The term “cosmeceutical agent” as used herein refers to a small molecule, nucleic acid, peptide, composition or other substance that provides a pharmacological, cosmeceutical, cosmetic, or any other beneficial activity. The term “active” as used herein refers to the ingredient, component or constituent of the compositions of the present invention responsible for the intended cosmeceutical effect. The terms “cosmeceutical agent” and “active agent” are used interchangeably. The active agent may be imidazolidinedione, an imidazaolinedione analog, or a chemical derived from an imidazolinedione analog (“derivative”) thereof.

As used herein, the phrase “additional active ingredient” refers to an agent, other than an imidazolidinedione analog compound of the inventive composition, or derivative thereof, that exerts a pharmacological, cosmeceutical, cosmetic, or any other beneficial activity.

It is to be understood that “other beneficial activity” may be one that is only perceived as such by the subject using the inventive compositions.

Compositions according to the described invention that further include one or more additional active ingredients therefore can be efficiently used, in addition to their use as a treatment for an epithelial-related condition, in the treatment of any medical, cosmetic, and/or cosmeceutical condition in which applying the additional active ingredient is beneficial.

Non-limiting examples of additional active ingredients according to the described invention include one or more, in any combination, of a protective agent, an emollient, an irritant, a keratolytic, a sun screening agent, a sun tanning agent, a peptide, niacinamide, farnesol, phytantriol, salicylic acid, hydroxy acid, an anti-cellulite agent, bisabolol, an antibiotic agent, an antifungal agent, an antiviral agent, an antiprotozoal agent, an anti-acne agent, a steroidal anti-inflammatory agent, a non-steroidal anti-inflammatory agent, an antipruritic agent, an anti-oxidant agent, an anti-histamine agent, a vitamin, an anti-wrinkle agent, an anti-skin atrophy agent, a sclerosing agent, a caustic agent and a hypo-pigmenting agent.

In the broadest pharmacological sense, a “protective” is any agent that isolates the exposed surface of the skin or other membrane from harmful or annoying stimuli. Protectives can be administered to the skin to form an adherent, continuous film that may be flexible or semi-rigid depending on the materials and the formulations as well as the manner in which they are applied. This material may serve several purposes including providing occlusion from the external environment, providing chemical support, and serving as vehicles for other medicaments. Examples include aluminum hydroxide gel, collodium, dimethicone, petrolatum gauze, absorbable gelatin film, absorbable gelatin sponge, zinc gelatin, kaolin, lanolin, anhydrous lanolin, mineral oil, mineral oil emulsion, mineral oil light, olive oil, peanut oil, petrolatum, silicones, hydrocolloids and the like.

In some embodiments, protectives included in the composition of the invention are demulcents. Demulcents are protective agents employed primarily to alleviate irritation, particularly of abraded tissues. They often are applied to the surface in a viscid, sticky preparation that covers the area readily and may be medicated. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like,

“Emollients” generally are bland, fatty or oleaginous materials that can be applied locally. Emollients increase the tissue moisture content, thereby rendering the skin softer and more pliable. Increased moisture content in the skin can be achieved by preventing water loss with an occlusive water-immiscible barrier, by increasing the water-holding capacity in the skin with humectants, or by altering the desquamation of the outermost skin layer, the stratum corneum. Useful emollients include lanolin, spermaceti, mineral oil, paraffin, petrolatum, white ointment, white petroleum, yellow ointment. Also included are vegetable oils, waxes, cetyl alcohol, glycerin, hydrophilic petrolatum, isopropyl myristate, myristyl alcohol, and oleyl alcohol.

An “irritant” is a material that acts locally on the skin to induce, based on irritant concentration, hyperemia, inflammation, and desiccation. Irritant agents include, but are not limited to, an alcohol, aromatic ammonia spirits, benzoin tincture, camphor capsicum, and coal tar extracts. In some embodiments, the irritant is a rubefacient. As used herein “rubefacients” are agents that induce hyperemia, wherein hyperemia means an increased amount of blood in a body part or organ. Rubefaction, which is induced by rubefacients, results from increased circulation to an injured area and is accompanied by a feeling of comfort, warmth, itching and hyperesthesia.

“Keratolytics” (desquamating agents) act to remove outer layers of the stratum corneum. This is particularly useful in hyperkeratotic areas. The keratolytics include benzoyl peroxide, fluorouracil, resorcinol, salicylic acid, tretinoin, and the like.

Representative examples of sun screening agents usable in context of the described invention include, without limitation, p-aminobenzoic acid and its salts and derivatives thereof (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-propylene glycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzylacetone and benzylacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinotine); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one) and 4-isopropyl-di-benzoylmethane, and any combination thereof.

Representative examples of sunless tanning agents usable in the described invention include, without limitation, dihydroxyacetone, glyceraldehyde, indoles and their derivatives. The sunless tanning agents can be used in combination with the sunscreen agents.

The compositions of the present invention also may contain an anti-cellulite agent. Suitable agents may include, but are not limited to, xanthine compounds, such as caffeine, theophylline, theobromine, and aminophylline.

Bisabolol is the primary active component of chamomile extract/oil. Bisabolol can be synthetic (d,1-alpha-isomer or (±)-alpha-isomer) or natural ((−)-alpha-isomer) in origin and can be used as essentially pure compounds or mixtures of compounds (e.g., extracts from natural sources such as chamomile). The alpha form of bisabolol (a-bisabolol) is used in a variety of cosmetic products as a skin conditioning or soothing agent. As used herein, “bisabolol” includes chamomile extract or oil and any isomers and tautomers of such. Suitable bisabolol compounds are commercially available as a natural material from Dragoco (Totowa, N.J.) under the product name alpha-bisabolol natural and as a synthetic material from Fluka (Milwaukee, Wis.) under the product name alpha-bisabolol. When present in the compositions of the present invention, the composition may contain from about 0.001% to about 50%, by weight of the composition, from about 0.01% to about 20%, from about 0.01% to about 15%, and from about 0.1% to about 10%, of bisabolol, from about 0.1% to about 5% of bisabolol.

Niacinamide (nicotinmide, nicotinic acid amide) is the amide of nicotinic acid (vitamin B₃). Niacinamide has demonstrated anti-inflammatory actions in patients with inflammatory skin conditions.

Farnesol is a naturally occurring substance which generally is believed to act as a precursor and/or intermediate in the biosynthesis of squalene and sterols, especially cholesterol. Farnesol also is involved in protein modification and regulation. Chemically, farnesol is [2E,6E]-3,7,11-trimethyl-2,6,10-dodecatrien-1-ol and as used herein the term “farnesol” includes isomers and tautomers of such. Farnesol is commercially available, e.g., under the names farnesol (a mixture of isomers from Dragoco, Totowa, N.J.) and trans-trans-farnesol (Sigma Chemical Company, St, Louis, Mo.). When present in the compositions of the present invention, the composition may contain from about 0.001% to about 50%, by weight of the composition, from about 0.01% to about 20%, from about 0.1% to about 15%, from about 0.1% to about 10%, from about 0.5% to about 5%, and from about 1% to about 5% of farnesol.

Phytantriol is the common name for the chemical known as 3,7,11,15, tetramethylhexadecane-1,2,3,-triol. Phytantriol is commercially available from BASF (Whyandotte, Mich.). For example, phytantriol is useful as a spider vessel/red blotchiness repair agent, a dark circle/puffy eye repair agent, sallowness repair agent, a sagging repair agent, an anti-itch agent, a skin thickening agent, a pore reduction agent, oil/shine reduction agent, a post-inflammatory hyperpigmentation repair agent, wound treating agent, an anti-cellulite agent, and regulating skin texture, including wrinkles and fine lines. When present in the compositions of the present invention, the composition may be contain an amount from about 0.001% to about 50% by weight of the composition, from about 0.01% to about 20%, from about 0.1% to about 15%, from about 0.2% to about 10%, from about 0.5% to about 10%, and from about 1% to about 5% phytantriol.

Hydroxy acids the may be used in the compositions of the described invention include salicylic acid and salicylic acid derivatives. When present in the compositions of the present invention, salicylic acid may be used in an amount of from about 0.01% to about 50%, from about 0.1% to about 20%, from about 0.1% to about 10%, from about 0.5% to about 5%, and from about 0.5% to about 2% by weight of the composition.

A “peptide” as used herein refers to any of various amides that are derived from two or more amino acids by combination of the amino group of one acid with the carboxyl group of another. Peptides are usually obtained by partial hydrolysis of proteins. A peptide may refer to a naturally occurring peptide, a peptide mimetic, and/or a synthetic peptide. Examples of peptides include, but are not limited to, naturally occurring pentapeptides, synthetic pentapeptides, derivatives of pentapeptides, and mixtures thereof. Additional peptides, including but not limited to, di-, tri-, and tetrapeptides and derivatives thereof, may be included in the compositions of the present invention in amounts that are safe and effective. As used herein, “peptides” refers to both the naturally occurring peptides and synthesized peptides. Also useful herein are naturally occurring and commercially available compositions that contain peptides. Suitable dipeptides for use herein include Carnosine (beta-ala-his). Suitable tripeptides for use herein include, gly-his-lys, arg-lys-arg, his-gly-gly. Some tripeptides and derivatives thereof include palmitoyl-gly-his-lys, which may be purchased as Biopeptide CL (100 ppm of palmitoyl-gly-his-lys commericaily available from Sederma, France); Peptide CK (arg-lys-arg); Peptide CK+(ac-arg-lys-arg-NH.sub.2); and a copper derivative of his-gly-gly sold commercially as lamin, from Sigma (St.Louis, Mo.). Suitable tetrapeptides for use herein include Peptide B, arg-ser-arg-lys. Additional peptides include palmitoyl-gly-his-lys, beta-ala-his, their derivatives, and combinations thereof, palmitoyl-gly-his-lys, their derivatives, and combinations thereof. When included in the present compositions, the composition may contain from about 1×10⁻⁶% to about 10%, from about 1×10⁻⁶% to about 0.1%, 1×10⁻⁵% to about 0.01% peptides, by weight of the composition. In some embodiments, the compositions contain from about 0.1% to about 5%, by weight of the composition, of such peptides as described herein. In another embodiment, the peptide-containing composition contains from about 0.1% to about 10% peptides, by weight of the composition.

The term “antibiotic agent” as used herein means any of a group of chemical substances having the capacity to inhibit the growth of, or to destroy bacteria, and other microorganisms, used chiefLy in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin ; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate ; Erythromycin ethyl succinate; Erythromycin glucoheptonate; Erythromycin lactobionate; Erytromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

The term “anti-fungal agent” as used herein means any of a group of chemical substances having the capacity to inhibit the growth of or to destroy fungi. Anti-fungal agents include but are not limited to Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, Pyrrolnitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconawle, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

The term “anti-viral agent” as used herein means any of a group of chemical substances having the capacity to inhibit the replication of or to destroy viruses used chiefly in the treatment of viral diseases. Anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, Zalcitabine, Zidovudine, Acemannan, Acetylleucine, Amantadine, Amidinomycin, Delavirdine, Foscamet, Indinavir, Interferon-β, Interferon-α, Interferon-γ, Kethoxal, Lysozyme, Methisazone, Moroxydine, Nevirapine, Podophyllotoxin, Ribavirin, Rimantadine, Ritonavir2, Saquinavir, Stailimycin, Statolon, Tromantadine, Zidovudine (AZT) and Xenazoic Acid.

The term “anti-protozoal agent” as used herein means any of a group of chemical substances having the capacity to inhibit the growth of or to destroy protozoans used chiefly in the treatment of protozoal diseases. Examples of antiprotozoal agents, without limitation include pyrimethamine (Daraprim®) sulfadiazine, and Leucovorin.

Suitable anti-acne agents of the described invention include, without limitation, keratolytics, such as salicylic acid, sulfur, glycolic, pyruvic acid, resorcinol, and N-acetylcysteine; and retinoids such as retinoic acid and its derivatives (e.g., cis and trans, esters).

“Steroidal anti-inflammatory agent”, as used herein, refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriameinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amccinafel, amcinafide, betamethasone and the balance of its esters, chloroprednisone, chlorprednisone acetate, clocortelone, clescinolone, dichiorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

“Non-steroidal anti-inflammatory agents” refers to a large group of agents that are aspirin-like in their action, including ibuprofen (Advil)®, naproxen sodium (Aleve)®, and acetaminophen (Tylenol)®. Additional examples of non-steroidal anti-inflammatory agents that are usable in the context of the described invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is useful for topical application.

The term “antipruritic agents” as used herein refers to those substances that reduce, eliminate or prevent itching. Suitable antipruritic agents include, without limitation, pharmaceutically acceptable salts of methdilazine and trimeprazine.

The term “anti-oxidant agent” as used herein refers to a substance that inhibits oxidation or reactions promoted by oxygen or peroxides. Non-limiting examples of anti-oxidants that are usable in the context of the described invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters (for example, propyl gallate), uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fiumaric acid and its salts, glycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

The term “antihistamine agent” as used herein refers to any of various compounds that counteract histamine in the body and that are used for treating allergic reactions (such as hay fever) and cold symptoms. Non-limiting examples of antihistamines usable in context of the described invention include chlorpheniramine, brompheniramine, dexchlorpheniramine, tripolidine, clemastine, diphenhydramine, promethazine, piperazines, piperidines, astemizole, loratadine and terfenadine.

The term “vitamin” as used herein, refers to any of various organic substances essential in minute quantities to the nutrition of most animal that act especially as coenzymes and precursors of coenzymes in the regulation of metabolic processes. Non-limiting examples of vitamins usable in context of the described invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), and vitamin B₃ (niacinamide and its derivatives).

“Anti-skin atrophy actives” refers to substances effective in replenishing or rejuvenating the epidermal layer by promoting or maintaining the natural process of desquamation. Examples of antiwrinkle and antiskin atrophy actives, which can be used in addition to the imidazolidinedione analog compounds of the described invention include, but are not limited to, retinoic acid, its prodrugs and its derivatives (e.g., cis and trans) and analogues; salicylic acid and derivatives thereof, sulfur-containing D and L amino acids and their derivatives and salts, particularly the N-acetyl derivatives (for example, N-acetyl L-cysteine); thiols, e.g. ethane thiol; alpha-hydroxy acids, e.g. glycolic acid,), alpha hydroxy acids (such as glycolic acid, lactic acid, tartaric acid, malic acid, citric acid, etc.) and beta hydroxy acids (such as salicylic acid and the like); lactic acid; phytic acid, lipoic acid; lysophosphatidic acid, and skin peel agents (e.g., phenol and the like).

“Caustic agents” refer to substances capable of destroying or eating away epithelial tissue by chemical action. Caustic agents can be used to remove dead skin cells. For example, beta-hydroxy acids, which are naturally derived acids with a strong kerolytic effect, are useful for problem skin, acne or peeling.

“Hypopigmenting agents” refer to substances capable of depigmenting the skin. Suitable hypopigmenting agents include hydroquinones, mequinol, and various protease inhibitors including serine protease inhibitors, active soy and retinoic acid.

The topical compositions of the described invention can be applied locally to the skin and may be in any form including solutions, oils, creams, ointments, gels, lotions, milks, moisturizers, sprays, and the like.

In another embodiment, an imidazolidinedione analog compound, carrier and, optionally, additional active ingredients are formed into a composition comprising a solution, emulsion or gel suspension.

In some embodiments, a composition comprising an imidazolidinedione analog compound, a pharmaceutical or cosmetic carrier and, optionally, one or more additional active ingredients is in the form of a solution. In another embodiment, an inventive composition comprising an imidazolidinedione analog compound, a carrier and other, optional ingredients can be dispersed in an emulsion. An emulsion is a two-phase system prepared by combining two immiscible liquid carriers, one of which is disbursed uniformly throughout the other and consists of globules that have diameters equal to or greater than those of the largest colloidal particles. The globule size is critical and must be such that the system achieves maximum stability. Usually, separation of the two phases will not occur unless a third substance, an emulsifying agent, is incorporated. Thus, a basic emulsion contains at least three components, the two immiscible liquid carriers and the emulsifying agent as well as the imidazolidinedione analog compound. Most emulsions incorporate an aqueous phase into a non-aqueous phase (or vice versa). However, it is possible to prepare emulsions that are basically non-aqueous, for example, anionic and cationic surfactants of the non-aqueous immiscible system glycerin and olive oil.

Emulsifying agent carriers useful in the described invention are described hereinabove.

In yet, another embodiment, the imidazolidinedione analogs of the inventive compositions can be mixed with a gel suspension, (a semi-solid carrier) or solid carrier to form a paste, powder, ointment, cream, lotion, hydrogel or the like. The term “paste” refers to a thick, stiff ointment containing at least 20% solids. “Powders” are fine particles that can result from comminution of any dry substance. Generally, powders consist of particles ranging in size from about 0.1μ to about 10,000μ, although the most useful pharmaceutical range is approximately from about 0.1μ to about 10μ. As used herein, the term “ointment” refers to a semisolid preparation intended to be applied externally to the skin. The term “cream” refers to an opaque, soft, cosmetically acceptable preparation intended for external application that comprises a water-soluble or cream base and that can be either a water-in-oil (w/o) or an oil in water (p/w) type of emulsion. The term “lotion” is used to designate solutions or suspensions that are applied topically. A solution generally is considered as a homogeneous mixture of two or more substances; it is frequently, though not necessarily, a liquid. In a solution, the molecules of the solute (or dissolved substance) are uniformly distributed among those of the solvent. A solution can be prepared by mixing a solute or dissolved substance (such as an imidazolidinedione analog compound of the invention and, optionally, one or more active ingredient(s)) uniformly throughout a solvent carrier such as water or organic solvents, such as the alcohols (e.g. ethanol or isopropanol, acetone). A suspension is a dispersion (mixture) in which a finely-divided species is combined with another species, with the former being so finely divided and mixed that it doesn't rapidly settle out. In everyday life, the most common suspensions are those of solids in liquid water.

“Gels” or “jellies” are semisolid systems consisting of suspensions made up of small inorganic particles or large organic molecules interpenetrated by a liquid. The concentration of the gelling agents in a gel typically is less than 10%. In some embodiments the concentration of the gelling agents in a gel is in the about 0.5% to about 2.0% range. A “hydrogel” is an extremely hydrated polymer gel wherein the polymer chain holds many times its weight in trapped water. It contains ingredients that are either dispersible as colloids or soluble in water, including organic hydrogels, natural and synthetic gums, and inorganic hydrogels. In high concentrations, hydrophilic colloids form semisolid gels, also referred to as jellies.

Examples of gelling agents include, but are not limited to, acacia, alginic acid, bentonite, carbomer, carboxymethylcellulose (CMC) sodium, cetostearyl alcohol, collloidal silicon dioxide, ethylcellulose, gelatin, guar gum, hydroxyethylcellulose, hydroxypropyl cellulose, hydroxypropyl methylcellulose, magnesium aluminum silicate, maltodextrin, methylcellulose, polyvinyl alchohol (PVA), povidone, propylene carbonate, propylene glycol alginate, sodium alginate, sodium starch glycolate, starch, tragacanth, and xanthan gum.

Alginic acid generally is used in concentrations between about 1% and about 5% as a thickening agent in gels. It swells in water to about 200 to about 300 times its own weight without dissolving. Cross-linking with increased viscosity occurs when adding a calcium salt, such as calcium citrate.

Carbomer (Carbopol®, a registered trademark of Noveon, Inc. (formerly B.F. Goodrich Co.)) resins are allyl pentaerythritol-cross-linked, acrylic acid-based polymers, which have a high molecular weight and are modified with C10 to C30 alkyl acrylates. They are fluffy, white, dry powders with large bulk densities, 2% maximum moisture, and pKa of 6.0±0.5. There are many carbomer resins, with viscosity ranges from 0 to 80,000 cP. pH is important in determining the viscosity of carbomer gels. Benzalkonium chloride (0.01%), sodium benzoate (0.01%), methylparaben (0.18%), propylparaben (0.02%), and thimersal (0.01%, 0.1%) are commonly used preservatives that are compatible with carbomer resins.

Carboxymethylcellulose (CMC) in concentrations of about 4% to about 6% of the medium viscosity grades can be used to produce gels; glycerin can be added to prevent drying. It is most stable at pH levels between 2 and 10.

Colloidal silicon dioxide can be used to prepare transparent gels when used with other ingredients of similar refractive index. It is most effective at pH values up to about 7.5.

Magnesium aluminum silicate (Veegum) in concentrations of about 10% forms firm, thixotropic gels. A thixotropic gel reversibly transitions from gel to sol on stressing and reverts back to a gel on standing. It should be noted that magnesium aluminum silicate can bind to some substances and limit their availability.

Plastibase/Jelene is a mixture of 5% low molecular weight polyethylene and 95% mineral oil. Polyoxamer, or Pluronic™, gels are made from selected forms of polyoxyethylene-polyoxypropylene copolymers in concentrations ranging from about 15% to about 50%. Because Pluronic F-127 has low toxicity and good solubilizing capacity and optical properties, it is generally considered to be a good medium for topical drug delivery systems.

Povidone, in the higher molecular weight forms, can be used to prepare gels in concentrations up to about 10%.

Propylene glycol alginate is used as a gelling agent in concentrations of about 1% to about 5%, depending on the specific application. The preparations are most stable at a pH of 3 to 6 and should contain a preservative.

Sodium alginate can be used to produce gels in concentrations up to about 10%. Aqueous preparations are most stable between pH values of about 4 to about 10. Sodium alginate has been used to produce gels that serve as ointment bases. In concentrations greater than about 2.5% and in the presence of soluble calcium salts, a firm gel, stable between pH 5 and pH 10, is formed. Sodium alginate gels for external use should be preserved, for example, with 0. 1% chloroxylenol or the parabens.

Tragacanth gum has been used to prepare gels that are most stable from pH 4 to about pH 8. These gels must be preserved, for example, with 0.1% benzoic acid or sodium bensoate or a combination of 0.17% methylparaben and 0.03% propylparaben. These gels can be sterilized by autoclaving.

Liqua-Gel™(Paddock), which contains purified water, propylene glycol, glycerin, hydroxypropyl methylcellulose, and potassium sorbate, is a liquid lubricating gel that is water soluble and nongreasy. It can be used to dissolve or suspend a variety of topically applied dermatologic agents. Sodium phosphate and boric acid are used to buffer the gel to a pH of about 5. Diazolidinyl urea and methyl paraben and propylparaben are included as preservatives.

In some embodiments, ointments may be prepared which are in gel-suspension form. These are semi-solid preparations intended for external application to the epithelium. Generally, ointment bases are categorized into hydrocarbon bases (oleaginous), which may use white petroleum as a base; adsorption bases (anhydrous), which might use hydrophilic petroleum or anhydrous lanolin; emulsion bases (water in oil type); emulsion bases (oil in water type); and water soluble bases, which often use polyethylene glycol as an ointment base. For example, if a water-in-oil emulsifying agent is added to an oleaginous base, an absorption base is formed. Examples of these bases include hydrophilic petrolatum, Aquabase, and Aquaphor. Ointments using an absorption base can be prepared by incorporation directly into the base or with the use of heat. Levigation (meaning to make into a smooth, fine powder or paste, as by grinding when moist) may be used to incorporate a water-insoluble powder. Oil-in-water emulsion bases, which are known as the “vanishing cream” types of preparations because they disappear, or vanish, on application, include Dermabase, Velvachol, and hydrophilic ointment USP. If an oil-in-water emulsion base is used, the levigating agent may be water, glycerin, propylene glycol, PEG 300 or 400, alcohol or some liquid miscible with water. A humectant in a concentration of about 2% to about 5% may be added to prevent an oil-in-water emulsion from drying too rapidly on the skin.

A cosmetic emulsion can be stabilized by using emulsifiers that complement the components of the formula. The hydrophilic-lipophilic balance (HLB) system may be used to determine the amounts of emulsifier(s) needed to stabilize an emulsion. Briefly, each ingredient of the formula, based upon its lipophilicity or lipophobicity, is assigned a value on a scale of 1 (very lipophilic) to 14 (very lipophobic). Ingredients with low BULB values require low HLB emulsifiers, while those with high HLB values require high HLB emulsifiers. The HLB value of a combination of different emulsifiers can be calculated as follows: HLB=((amount of first emulsifier*HLB of first emulsifier)+(amount of second emulsifer*HLB of second emulsifier))/(amount of first emulsifier+amount of second emulsifier). As always, because trial and error is necessary in order to stabilize a specific emulsion system, the HLB system is used as a starting point.

For example, emulsifiers in the Span(t series are oil-soluble. Accordingly, their oil soluble character will cause the oil phase to predominate and form a water-in-oil emulsion. Examples of the Span® series of emulsifiers include, but are not limited to, Span20®, sorbitan laurate or sorbitan monododeconoate. The emulsfiers in the Tween® series are water soluble. Accordingly, their water soluble character will cause the water phase to predominate and form an oil-in-water emulsion. Examples of the Tween® series of emulsifiers include, but are not limited to, Tween20® and Tween80®.

Additional compositions using the imidazolidinedione analog compounds of the described invention and carriers can be readily prepared using technology which is known in the art such as described in Remington's Pharmaceutical Sciences, 18^{th} or 19^{th} editions, published by the Mack Publishing Company of Easton, Pennsylvania, and in Loyd v. Allen Jr., The Art, Science, and Technology of Pharmaceutical Compounding, 2d Edition, published by the American Pharmaceutical Association (2002), the contents of which are expressly incorporated herein by reference.

The process to prepare the compositions described hereinabove generally includes admixing the at least one imidazolidinedione analog compound, as described hereinabove, and the pharmaceutically, cosmetically or cosmeccutically acceptable carrier. In cases where additional active ingredients, as detailed above, are present in the compositions, the process includes admixing these ingredients together with the active ingredients and the carrier. The mixing technique utilized in the process of the described invention can involve any one of the known techniques for formulating topical compositions. A variety of exemplary formulation techniques that are usable in the process of the described invention is described, for example, in Harry's Cosmeticology, Seventh Edition, Edited by J B Wilkinson and R J Moore, Longmann Scientific & Technical, 1982, which is incorporated herein by reference.

According to another aspect of the described invention, there is provided a method of treating wrinkles. The method is effected by topically applying a pharmaceutically, cosmetically or cosmeceutically effective amount of the composition of the described invention as described above onto an epithelial surface.

According to one embodiment, the described invention provides a method of treating or preventing wrinkling of skin, the method comprising the step of topically applying onto an epithelial surface of a subject, including a human, in need thereof, a cosmetically effective amount of a composition comprising (a) a compound of formula J, or a salt of formula I, wherein R¹, R², R³, R⁴, and R⁵ are each independently H, alkyl, cycloalkyl, alkylcycloalkyl, alkylaryl, alkylheteroalkyl; A, B and C are each independently C, N, S, O, SO or SO₂; X═CH═CH; C, NR⁵, O, S or a bond to the phenyl ring; Y, is halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵; n=0, 1, 2, 3, 4, or 5; Zₘ=halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵; m=0, 1, 2, or 3; and (b) a carrier thereby treating or preventing wrinkles.

As used herein the terms “pharmaceutically effective amount” “cosmetically effective amount”, “wrinkle-reducing amount”, or “cosmeceutically effective amount” refer to the amount of any of the compositions of the invention that result in a cosmeceutical or beneficial effect following its administration to a subject. The pharmaceutical, cosmeceutical or cosmetic effect can be curing, minimizing, preventing or ameliorating a skin condition, improving the physical appearance and aesthetics, or may have any other pharmaceutical, cosmeceutical or cosmetic beneficial effect. The concentration of the substance is selected so as to exert its pharmaceutical, cosmeceutical or cosmetic effect, but low enough to avoid significant side effects within the scope and sound judgment of the skilled artisan. The effective amount of the composition may vary with the particular epithelial tissue being treated, the age and physical condition of the biological subject being treated, the severity of the condition, the duration of the treatment, the nature of concurrent therapy, the specific compound, composition or other active ingredient employed, the particular carrier utilized, and like factors.

A skilled artisan can determine a cosmeceutically effective amount of the inventive compositions by determining the unit dose. As used herein, a “unit dose” refers to the amount of inventive composition required to produce a response of 50% of maximal effect (i.e. ED₅₀). The unit dose can be assessed by extrapolating from dose-response curves derived from in vitro or animal model test systems. Generally, the compositions of the described invention include about 0.01% to about 10.0% w/w of at least one imidazolidinedione analog compound. The compositions of the described invention are applied topically. For some subjects, the compositions of the described invention are applied topically once daily. For some subjects, the compositions of the described invention are applied topically. For some subjects, the compositions of the described invention are applied topically as directed by a physician. For some subjects, topical application may be required periodically for an indeterminate length of time.

According to another embodiment, the epithelial surface onto which the composition is applied topically is a face. According to another embodiment, the epithelial surface onto which the composition is applied topically comprises skin around the eye. According to another embodiment, the epithelial surface onto which the composition is applied topically comprises an eyelid. According to another embodiment, the epithelial surface onto which the composition is applied topically comprises skin around a mouth. According to another embodiment, the epithelial surface onto which the composition is applied topically comprises a forehead. According to another embodiment, the epithelial surface onto which the composition is applied topically comprises a decolletage. According to another embodiment, the epithelial surface onto which the composition is applied topically comprises a neck.

According to another embodiment, the compound of formula I is dantrolene, or a salt thereof. According to another embodiment, the compound of formula I is azumolene, or a salt thereof.

According to another embodiment, the composition farther includes an additional active ingredient. According to some such embodiments, the additional active ingredient is one or more, in any combination, of a protective agent, an emollient, an irritant, a keratolytic, a sun screening agent, a sun tanning agent, a peptide, a niacinamide, farnesol, phytantriol, salicylic acid, hydroxy acid, an anti-cellulite agent, bisabolol, an antibiotic agent, an antifungal agent, an antiviral agent, an antiprotozoal agent, an anti-acne agent, a steroidal anti-inflammatory agent, a non-steroidal anti-inflammatory agent, an antipruritic agent, an anti-oxidant agent, an anti-histamine agent, a vitamin, an anti-wrinkle agent, an anti-skin atrophy agent, a sclerosing agent, a caustic agent and a hypo-pigmenting agent.

According to another embodiment, the composition is formulated as a cream. According to another embodiment, the composition is formulated as a lotion. According to another embodiment, the composition is formulated as an ointment. According to another embodiment, the composition is formulated as a gel.

As used herein the term “treating” includes abrogating, substantially inhibiting, slowing or reversing the progression of a condition, substantially ameliorating clinical or aesthetical symptoms of a condition, substantially preventing the appearance of clinical or aesthetical symptoms of a condition, protecting from harmful or annoying stimuli or generally promoting healthy epithelial tissue.

The term “condition” includes a variety of conditions, including the promotion of healthy epithelium or skin. As used herein “promotion of healthy skin” or “promoting healthy skin”, refers to providing cooling or soothing sensations, or reducing puffiness, or promoting the appearance of reduced wrinkling or puffiness. This phrase also refers to the subject's perception of his/her skin as appearing healthy or having the perception of wellness or youth.

The term “subject” as used herein includes animals of mammalian origin. In some embodiments, the subject is a human.

The term “reduce” or “reducing” as used herein refers to the act of limiting the occurrence of a disorder, a trait, a symptom, a wrinkle, or other such event or characteristic or condition in individuals at risk of developing such a particular disorder, trait, symptom, wrinkle or other such event, characteristic or condition.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight temperature is in degrees Centigrade, and pressure is at or near atmospheric.

### Example 1

### Crow's Feet

A subject showing undesirable crow's feet wrinkling around the eye is treated by topical application of a wrinkle-reducing amount of a composition of the described invention comprising at least one imidazolidinedione analog compound. For some subjects, the composition is applied once daily to decrease the appearance of crow's feet in such subjects. For some subjects, the composition is applied twice daily to decrease the appearance of crow's feet in such subjects. For some subjects, the composition is applied as directed by a physician to decrease the appearance of crow's feet in such subjects.

### Example 2

### Undereye Wrinkles

A subject showing undesirable undereye wrinkles around the eye is treated by topical application of a wrinkle-reducing amount of a composition of the described invention comprising at least one imidazolidinedione analog compound. For some subjects, the composition is applied once daily to decrease the appearance of undereye wrinkles in such subjects. For some subjects, the composition is applied twice daily to decrease the appearance of undereye wrinkles in such subjects. For some subjects, the composition is applied as directed by a physician to decrease the appearance of undereye wrinkles in such subjects.

### Example 3

### Lip Wrinkles

A subject showing undesirable wrinkles around the lip area is treated by topical application of a wrinkle-reducing amount of a composition of the described invention comprising at least one imidazolidinedione analog compound, For some subjects, the composition is applied once daily to decrease the appearance of lip wrinkles in such subjects. For some subjects, the composition is applied twice daily to decrease the appearance of lip wrinkles in such subjects. For some subjects, the composition is applied as directed by a physician to decrease the appearance of lip wrinkles in such subjects.

### Example 4

### Forehead Wrinkles

A subject showing undesirable forehead wrinkles is treated by topical application of a wrinkle-reducing amount of a composition of the described invention comprising at least one imidazolidinedione analog compound. For some subjects, the composition is applied once daily to decrease the appearance of forehead wrinkles in such subjects. For some subjects, the composition is applied twice daily to decrease the appearance of forehead wrinkles in such subjects. For some subjects, the composition is applied as directed by a physician to decrease the appearance of forehead wrinkles in such subjects.

### Example 4

### Neck Wrinkles

A subject showing undesirable neck wrinkles is treated by topical application of a wrinkle-reducing amount of a composition of the described invention comprising at least one imidazolidinedione analog compound. For some subjects, the composition is applied once daily to decrease the appearance of neck wrinkles in such subjects. For some subjects, the composition is applied twice daily to decrease the appearance of neck wrinkles in such subjects. For some subjects, the composition is applied as directed by a physician to decrease the appearance of neck wrinkles in such subjects.

### Example 5

### Decolletage Wrinkles

A subject showing undesirable neck wrinkles is treated by topical application of a wrinkle-reducing amount of a composition of the described invention comprising at least one imidazolidinedione analog compound. For some subjects, the composition is applied once daily to decrease the appearance of neck wrinkles in such subjects. For some subjects, the composition is applied twice daily to decrease the appearance of neck wrinkles in such subjects. For some subjects, the composition is applied as directed.

### Example 6

### Imidazolidinedione 4% Liquid-Solid Emulsion Gel

Liquid-solid emulsion gels can be prepared from gelatin and a selection of an alcohol from a homologous series (e.g., octanol, nonanol, decanol, undecanol, or dodecanol). A imidazolidinedione 4% liquid-solid emulsion gel is formulated. The gelatin solution contains Gelatin, 200 bloom (8 g) and phosphate buffer (pH 7) (40 ml). The gel product contains gelatin solution (40 ml) and long-chain alcohol (10 g).

Briefly, an the aqueous gelatin base such as 20% (w/w) 200 bloom gelatin is formulated in phosphate buffer (pH 7). The gelatin-water mixture is matured for about 1 hour at room temperature (approximately 25° C.) and then is melted at 60° C. The molten gel is left at 60° C. for another 2 hours to allow air bubbles to escape. 10 g of the long-chain alcohol, which has been preheated to 60° C., is added to 40 g of the heated molten aqueous gel, and stirred at high speed for about 2 minutes. The active (4 g per 100 ml) is added to the appropriate phase. The molten mixture is poured onto a plate or between two plates to set or “cast.” The circular or other shaped portions of the gel are cut out and applied to the skin to release the enclosed drug.

### Example 7

### Methylcellulose Gels

Methylcellulose gels can be prepared from methylcellulose, water and the active ingredient. The methylcellulose gel contains methylcellulose 1500 cP (1%-5%) and purified water.

Briefly, the methylcellulose is added to about 50 mL of boiling purified water and dispersed well. The remaining purified water, ice cold, is added to bring the volume of the gel to 100 ml. The active ingredient(s) then are added. The mixture is stirred until uniform and thickened.

### Example 8

### White Ointment

A white ointment containing the active ingredient can be prepared. The ointment contains white wax (50 g) and white petrolatum (950 g). Briefly, the white wax is placed in a suitable container and melted using a water bath. The white petrolatum is added and mixed until uniform, and cooled. Before the mixture solidifies, the active ingredient is added.

### Example 9

### Cold Cream

A cold cream containing the active ingredient can be prepared. The cold cream contains cetyl esters wax (125 g), white wax (120 g), mineral oil (560 g), sodium borate (5 g) and purified water (190 mL). Briefly, reduce the cetyl esters wax and the white wax are reduced to small pieces; the pieces are melted using a water bath. The mineral oil is added and the mixture heated until it reaches 70° C. Sodium borate is dissolved in the purified water, which has been warmed to 70° C. The warm mixture is added gradually to the melted oleaginous mixture. The mixture is removed from the heat and stirred rapidly and continuously. The active ingredient(s) are added until the mixture has congealed.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A topical composition to treat or prevent skin wrinkling comprising.
(a) a cosmeceutically effective amount of a compound of formula I or a salt of Formula I: 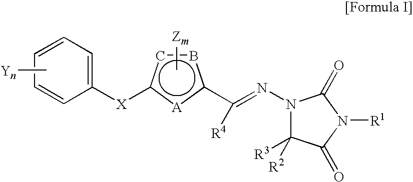
wherein
R¹, R², R³, R⁴, and R⁵ are each independently H, alkyl, cycloalkyl, alkylcycloalkyl, alkylaryl, alkylheteroalkyl;
A, B and C are each independently C, N, S, O, SO or SO₂;
X═CH═CH; C, NR⁵, O, S or a bond to the phenyl ring;
Yₙ is halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵;
n=0, 1, 2, 3, 4, or 5;
Zₘ=halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵; m=0, 1, 2, or 3; and
(b) a carrier.

2. The composition according to claim 1, wherein the compound of formula I is dantrolene.

3. The composition according to claim 1, wherein the compound of formula I is azumolene.

4. The composition according to claim 1, wherein the composition is applied to skin around at least one of an eye, a mouth, a nose, a forehead, a scalp, a decolletage, and a neck.

5. The composition according to claim 1, wherein the composition further includes an additional active ingredient.

6. The composition according to claim 5, wherein the additional active ingredient is selected from the group consisting of a protective agent, an emollient, an irritant, a keratolytic, a sun screening agent, a sun tanning agent, a peptide, a niacinamide, farnesol, phytantriol, salicylic acid, hydroxy acid, an anti-cellulite agent, bisabolol, an antibiotic agent, an antifungal agent, an antiviral agent, an antiprotozoal agent, an anti-acne agent, a steroidal anti-inflammatory agent, a non-steroidal anti-inflammatory agent, an antipruritic agent, an anti-oxidant agent, an anti-histamine agent, a vitamin, an anti-wrinkle agent, an anti-skin atrophy agent, a sclerosing agent, a caustic agent and a hypo-pigmenting agent, or combinations thereof.

7. The composition according to claim 1, wherein the composition is formulated as a cream, a lotion, an ointment or a gel.

8. A method of treating or preventing wrinkling of skin, the method comprising the step of topically applying onto an epithelial surface of a subject, including a human, in need thereof, a cosmeceutically effective amount of a composition comprising
(a) a compound of Formula I or a salt of formula I: 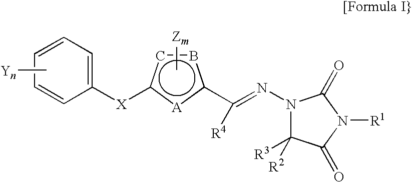
wherein
R¹, R², R³, R⁴, and R⁵ are each independently H, alkyl, cycloalkyl, alkylcycloalkyl, alkylaryl, alkylheteroalkyl;
A, B and C are each independently C, N, S, O, SO or SO₂;
X═CH═CH; C, NR⁵, O, S or a bond to the phenyl ring;
Yₙ is halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵;
n=0, 1, 2, 3, 4, or 5;
Zₘ=halogen, alkyl, haloalkyl, alkoxy, alkylenyl; alkenylenyl; carbamyl; cycloalkyl; cycloalkylenyl; heterocylcyl, arylenyl; heteroaryl; heteroarylenyl; nitro; OR¹, OR², OR³, OR⁴, OR⁵, NR¹, NR², NR³, NR⁴, NR⁵, CONR¹, CONR², CONR³, CONR⁴, CONR⁵; m=0, 1, 2, or 3; and
(b) a carrier
thereby treating or preventing wrinkles.

9. The method according to claim 7, wherein the compound of formula I is dantrolene.

10. The method according to claim 7, wherein the compound of formula I is azumolene.

11. The method according to claim 7, wherein the epithelial surface onto which the composition is applied topically is a face.

12. The method according to claim 7, wherein the epithelial surface onto which the composition is applied topically comprises skin around an eye.

13. The method according to claim 7, wherein the epithelial surface onto which the composition is applied topically comprises an eyelid.

14. The method according to claim 7, wherein the epithelial surface onto which the composition is applied topically comprises skin around a mouth.

15. The method according to claim 7, wherein the epithelial surface onto which the composition is applied topically comprises a forehead.

16. The method according to claim 7, wherein the epithelial surface onto which the composition is applied topically comprises a decolletage.

17. The method according to claim 7, wherein the epithelial surface onto which the composition is applied topically comprises a neck.

18. The method according to claim 7, wherein the composition further includes an additional active ingredient.

19. The method according to claim 18, wherein the additional active ingredient is selected from the group consisting of a protective agent, an emollient, an irritant, a keratolytic, a sun screening agent, a sun tanning agent, a peptide, a niacinarnide, farnesol, phytantriol, salicylic acid, hydroxy acid, an anti-cellulite agent, bisabolol, an antibiotic agent, an antifungal agent, an antiviral agent, an antiprotozoal agent, an anti-acne agent, a steroidal anti-inflammatory agent, a non-steroidal anti-inflammatory agent, an antipruritic agent, an anti-oxidant agent, an anti-histamine agent, a vitamin, an anti-wrinkle agent, an anti-skin atrophy agent, a sclerosing agent, a caustic agent and a hypo-pigmenting agent, or combinations thereof.

20. The method according to claim 7, wherein the composition is formulated as a cream, a lotion, an ointment, or a gel.
